(19) United States
(12) Patent Application Publication
(10) Pub. No.: US 2007/0185517 A1
(43) Pub. Date: Aug. 9, 2007

(54) Method of securing acupuncture needle to insertion tube and acupuncture needle with insertion tube

(75) Inventor: Ryoichi Nakano, Shizuoka (JP)

Correspondence Address: MCDERMOTT WILL & EMERY LLP, 18191 VON KARMAN AVE., SUITE 500, IRVINE, CA, 92612-7108, US

(73) Assignee: SEIRIN CORPORATION, Shizuoka (JP)

(21) Appl. No.: 11/723,057

(22) Filed: Mar. 16, 2007

Related U.S. Application Data
(63) Continuation of application No. PCT/JP04/19057, filed on Dec. 21, 2004.

(51) Int. Cl. A61B 17/34

(52) U.S. Cl. 606/189

(57) ABSTRACT

There is provided an acupuncture needle for use in acupuncture treatment. The acupuncture needle having a metal needle handle is secured within an insertion tube made of a resin. A resin in an area where a surface of a needle handle 2 of an acupuncture needle 1 and an inner surface of an insertion tube 3 are in contact with each other is melted by high-frequency-induction heating or microwave heating, whereby the two are secured to each other. At this time, there is no need to use another member and thus the two can be secured to each other without increasing the number of components. In addition, by holding the insertion tube 3 and tapping an end portion of the needle handle 2, the acupuncture needle 1 can be released from the insertion tube 3 with one hand.

This application is a continuation of PCT/JP2004/019057, filed on Dec. 21, 2004.

### BACKGROUND OF THE INVENTION

1. Field of the Invention

The present invention relates to an acupuncture needle for use in acupuncture treatment, and particularly to a method of securing an acupuncture needle having a metal needle handle within a resin insertion tube, and an acupuncture needle with an insertion tube.

2. Description of the Related Art

Conventionally, various acupuncture needles for use in acupuncture treatment have been proposed. Upon performing acupuncture treatment, it is common to insert an acupuncture needle using a cylindrical insertion tube. As an acupuncture needle as a commercial product, there exists an acupuncture needle with an insertion tube, in which the acupuncture needle is contained in the insertion tube and thereby a needle tip thereof is protected. In such an acupuncture needle with the insertion tube, it is necessary that a needle handle be secured to an inner surface of the insertion tube with a portion of the needle handle protruding from an opening end of the insertion tube, and upon acupuncture treatment the needle be easily released and detached from the insertion tube.

Therefore, as a conventional general acupuncture needle, there exists an acupuncture needle in which by press-fitting a wedge-shaped chip made of another member into an interspace between a needle handle thereof and a needle tube, the two are secured to each other.

As another acupuncture needle, there exists an acupuncture needle in which a needle handle thereof and a needle tube are formed of the same resin and an ultrasonic vibration as well as a pressure are applied to generate intense friction heat in part of the resin, whereby portions to be jointed together are melted and the two are secured to each other (refer to Japanese Unexamined Patent Publication No. 1996(H08)-10310, and Japanese Unexamined Patent Publication No. 1997(H09)-28758).

As still another acupuncture needle, there exits an acupuncture needle in which a needle handle thereof and a needle tube are bonded together with an adhesive, whereby the two are fixedly secured to each other (refer to Japanese Unexamined Patent Publication No. 1996(H08)-155006).

However, in the method of securing an acupuncture needle to an insertion tube using a chip made of another member, there are problems that the number of components must increase, and moreover, upon acupuncture treatment, the chip first needs to be removed and thus it takes a lot of trouble with removal of the chip, and the acupuncture needle and the insertion tube easily come apart.

Further, in the securing method by ultrasonic welding described in the patent references 1 and 2, when a needle handle and an insertion tube are made of the same resin, the two can be welded and secured to each other; however, when, for example, the two are made of different materials, particularly when a needle handle of an acupuncture needle is made of a metal and an insertion tube is made of a resin, it is difficult to weld the two together.

Furthermore, in the case of the securing method by means of an adhesive described in the patent reference 3, even if a needle handle of an acupuncture needle and an insertion tube are of different materials, the two can be secured to each other; however, there are problems that control upon manufacturing is difficult and a lot of cumbersome works has been required.

### SUMMARY OF THE INVENTION

Therefore, taking consideration the above-described problems encountered up to now, an object of the present invention is to provide a method of securing an acupuncture needle having a metal needle handle within a resin insertion tube without increasing the number of components, and an acupuncture needle with an insertion tube.

According to a first aspect of the present invention, there is provided a method of securing an acupuncture needle to an insertion tube, comprising the steps of: inserting the acupuncture needle having a needle handle made of any one of materials including a metal and a resin, into the insertion tube made of the other of the materials different from that of the needle handle; arranging the insertion tube in a heating coil; feeding a high-frequency alternating current to the heating coil to induction-heat one of the needle handle and the needle tube, either of which is made of a metal; and melting, by heat, a resin in an area where a surface of the needle handle and an inner surface of the insertion tube are in contact with each other, to thereby secure the two to each other.

According to a second aspect of the present invention, there is provided a method of securing an acupuncture needle to an insertion tube, comprising the steps of: inserting the acupuncture needle having a metal needle handle into the insertion tube made of a resin; arranging an electrode at a position outside the needle tube and connecting another electrode to the needle handle; applying irradiation of a microwave onto the insertion tube disposed between the electrodes to thereby allow an irradiated area to be heated; and melting, by heat, an inner surface of the insertion tube to secure the needle handle.

According to a third aspect of the present invention, there is provided an acupuncture needle with an insertion tube in which the acupuncture needle having a needle handle made of any one of materials including a metal and a resin, is inserted into the insertion tube made of the other of the materials different from that of the needle handle, thereby secure the two to each other, wherein one of the needle handle and the insertion tube, either of which is made of a metal, is induction-heated by high-frequency induction heating to heat and melt a resin in an area where a surface of the needle handle and an inner surface of the insertion tube are in contact with each other, thereby effecting fixing of the two to each other.

Here, the one of the needle handle and the insertion tube, either of which is made of a metal, has, formed thereon, concave portions or convex portions annularly-arranged in a circumferential direction of a surface thereof.

In addition, the one of the needle handle and the insertion tube, either of which is made of a metal, may have, formed on a surface thereof, concave portions or convex portions that extend in an axial direction thereof.

Further, the one of the needle handle and the insertion tube, either of which is made of a metal, may have, irregularly formed on a surface thereof, concave portions or convex portions in a dotted fashion.

According to a fourth aspect of the present invention, there is provided an acupuncture needle with an insertion tube in which the acupuncture needle having a metal needle handle is inserted into the insertion tube made of a resin, to thereby secure the two to each other, wherein the insertion tube is heated by microwave heating and an inner surface of the insertion tube is melted by the heat, thereby effecting securing of the needle handle.

According to the present invention, even when a needle handle of an acupuncture needle is made of a metal and an insertion tube that contains the acupuncture needle is made of a resin, a resin in an area where a surface of the needle handle and an inner surface of the insertion tube are in contact with each other is melted by high-frequency induction heating or microwave heating, so that the two can be secured to each other.

At this time, there is no need to use another member and thus, the two can be secured to each other without increasing the number of components. In addition, by holding the insertion tube and tapping an end portion of the needle handle, the acupuncture needle can be released and detached from the insertion tube with one hand.

### BRIEF DESCRIPTION OF THE DRAWINGS

FIG. 1 is a partial cross-sectional view of an acupuncture needle with an insertion tube, showing an example of an embodiment of the present invention.

FIG. 2 is a view taken along an arrow A of FIG. 1.

FIG. 3 is a schematic view showing a first method of securing an acupuncture needle to an insertion tube, according to the present invention.

FIG. 4 is a perspective view, in part enlarged, showing a main part of a second shape of an acupuncture needle.

FIG. 5 is a cross-sectional view, in part enlarged, showing a main part of a third shape of an acupuncture needle.

FIG. 6 is a cross-sectional view, in part enlarged, showing a main part of a fourth shape of an acupuncture needle.

FIG. 7 is a perspective view, in part enlarged, showing a main part of a fifth shape of an acupuncture needle.

FIG. 8 is a perspective view, in part enlarged, showing a main part of a sixth shape of an acupuncture needle.

FIG. 9 is a schematic view showing a second method of securing an acupuncture needle to an insertion tube, according to the present invention.

### DESCRIPTION OF THE PREFERRED EMBODIMENTS

Hereunder is a detailed description of the present invention based on the accompanying drawings.

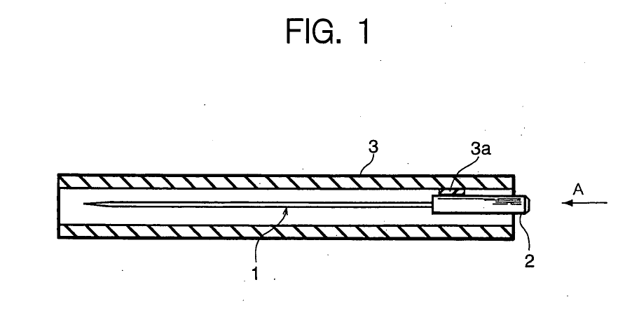

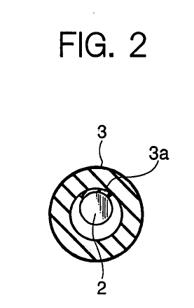

FIGS. 1 and 2 show a first embodiment of the present invention, FIG. 1 is a partial cross-sectional view of an acupuncture needle with an insertion tube, and FIG. 2 is a view on arrow A of FIG. 1. The acupuncture needle with the insertion tube is such that a needle handle 2 of an acupuncture needle 1 is secured at a predetermined area 3a within an insertion tube 3.

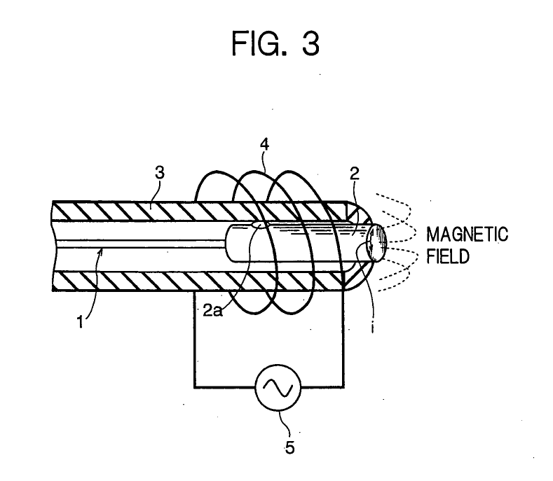

The acupuncture needle 1 has a metal rod-like body having a needle tip formed at a tip end side and the needle handle 2 is mounted on a base end side of the body. The needle handle 2 has a cylindrical body made of a metal such as a stainless-steel, for example, and as shown in FIG. 3, a single protruding portion 2a is formed on a surface of the needle handle 2.

The insertion tube 3 has a cylindrical-resin body having an inner diameter greater than an outer diameter of the needle handle 2. For a resin material, a resin such as polypropylene, polyethylene, polystyrene, or vinyl chloride, for example, is used.

Next, a method of securing such an acupuncture needle 1 having the metal needle handle 2 within the resin insertion tube 3 will be described. First, as shown in FIG. 3, the acupuncture needle 1 is inserted into the insertion tube 3 with part of the needle handle 2 protruding from an opening end of the insertion tube 3, and the insertion tube 3 is arranged in a heating coil 4. The heating coil 4 is, for example, an electrically conducting wire wound spirally around the insertion tube 3 such that the diameter of the spiral is greater than the outer diameter of the insertion tube 3, and is connected to an alternating-current power supply 5.

In this state, when a high-frequency alternating current is fed to the heating coil 4, a magnetic field is generated inside the heating coil 4. Since the magnetic field is also generated in the metal needle handle 2, a high-frequency eddy-current (circulating current) i is induced in the needle handle 2 in a direction in which the magnetic field is canceled out. When the eddy-current i flows through the needle handle 2, Joule heat is generated from the surface of the needle handle 2. Note that since the resistance value of the protruding portion 2a on the surface of the needle handle 2 increases, the heating temperature of the protruding portion 2a further increases.

In the case of such a high-frequency-induction-heating method, despite the fact that the heating coil 4 and the metal needle handle 2 are not in contact with each other, the entire surface of the needle handle 2 can be momentarily heated, and by the heating of the needle handle 2, an inner surface of the resin insertion tube 3 being in contact with the protruding portion 2a on the surface of the needle handle 2 is melted, whereby the two can be secured to each other at the melted area 3a (refer to FIG. 1).

Note that the shape of the heating coil 4 is not limited to a spiral shown in FIG. 3 and any shape can be used as long as the shape allows a magnetic field to be generated inside the heating coil 4 when a high-frequency alternating current is fed. For example, one obtained by bending a single circular wound coil into a half circle may be used. The heating temperature of the needle handle 2 can be arbitrarily set by appropriately selecting the number of windings or shape of the heating coil 4, the frequency of an alternating current, or the material of the needle handle 2. Particularly, by controlling the frequency of the alternating current power supply 5 by an electric control circuit, the heating temperature of the needle handle 2 can be easily set.

In the acupuncture needle with the insertion tube thus manufactured, the acupuncture needle 1 never be detached from the insertion tube 3 by vibration or the like during testing, packaging, transporting, or the like, before being used for acupuncture treatment. Upon acupuncture treatment, by holding the insertion tube 3 and tapping an end portion of the needle handle 2, the acupuncture needle 1 can be released and detached from the insertion tube 3 with one hand.

Although in the above description the single protruding portion 2a is formed on the surface of the metal needle handle 2, the present invention is not limited thereto and arbitrary recesses and projections may be formed on the surface of the needle handle 2. This will be described below.

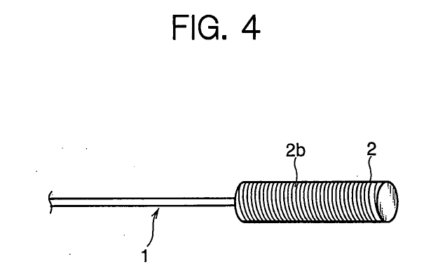

FIG. 4 is a perspective view, in part enlarged, showing a main part of a second shape of the aforementioned acupuncture needle 1. In the acupuncture needle 1, a plurality of groove portions 2bannularly-arranged in a circumferential direction on a surface of a metal needle handle 2 are formed. When such an acupuncture needle 1 having the needle handle 2 having formed therein the groove portions 2b is inserted into an insertion tube 3, the surface of the needle handle 2 and an inner surface of the insertion tube 3 come into contact with each other at multipoint. Thus, when the metal needle handle 2 is heated by high-frequency induction heating, the multipoint at which the needle handle 2 and the insertion tube 3 are in contact with each other become melted areas and therefore, the two can be more strongly secured to each other.

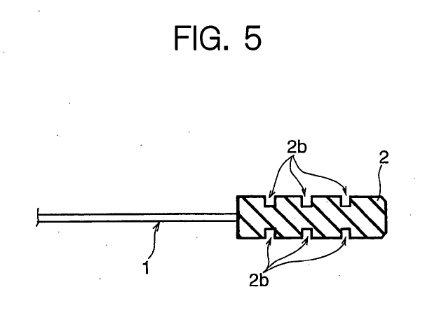

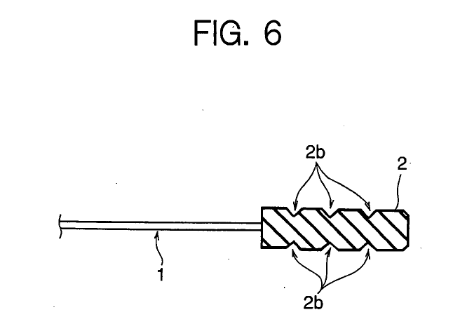

Note that the groove portions 2bformed in the needle handle 2 can be of any shape. For example, as shown in FIG. 5, the shape may be such that a cross section of a needle handle 2 is rectangular concaved, or as shown in FIG. 6, the shape may be such that the cross section is triangular concaved. Here, when, as shown in FIG. 5, rectangular-shaped groove portions 2b are formed in a surface of the needle handle 2, a resin is melted with the surface of the needle handle 2 and an inner surface of the insertion tube 3 being in contact with each other and the melted resin also enters into the rectangular-shaped groove portions 2b, and thus, stronger securing can be obtained as compared with that obtained with the shape shown in FIG. 6.

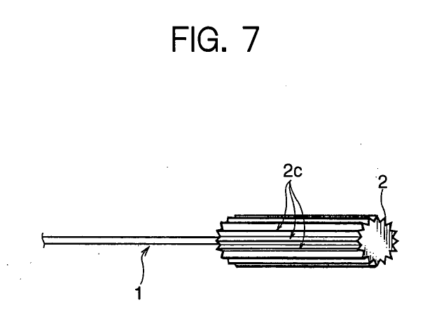

Further, as shown in FIG. 7, a plurality of chevron portions 2c extending in an axial direction of a needle handle 2 may be formed on a surface of the needle handle 2. In this case, when adjacent chevron portions 2c are formed in the same shape and at the same pitch, even after an acupuncture needle 1 is released from an insertion tube 3 once, the acupuncture needle 1 can be mounted in the insertion tube 3 again.

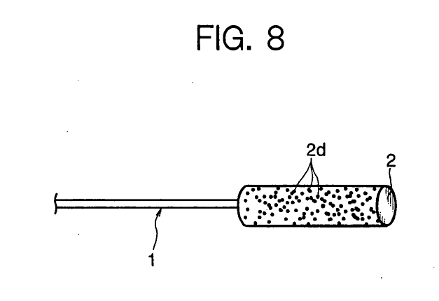

Furthermore, as shown in FIG. 8, a needle handle 2 made of a metal may be subjected to a blasting process or a process such as barreling or electrolytic polishing for example, to irregularly form concave portions (or convex portions) 2d in a dotted fashion on a surface of the needle handle 2. In this case, by high-frequency induction heating, the needle handle 2 of an acupuncture needle 1 and an insertion tube 3 are secured to each other at the irregularly formed concave portions 2d. Therefore, the acupuncture needle 1 once released from the insertion tube 3 cannot be mounted in the insertion tube 3 again and accordingly an acupuncture needle with an insertion tube intended for single-use only can be manufactured.

Although in the above description, for example, the protruding portion 2a, the groove portions 2b, the chevron portions 2c, the concave portions 2d, or the like, are formed on the surface of the needle handle 2, the present invention is not limited thereto and the shape, arrangement, and the like, of convex and concave portions may be set in any manner. For example, convex and concave portions may be formed in an oblique direction of the surface of the needle handle 2 or in a spiral fashion. Furthermore, without forming convex and concave portions on the needle handle 2, even when the surface of the needle handle 2 is smooth, the needle handle 2 can be similarly secured within the insertion tube 3.

Although here the method of securing an acupuncture needle 1 having a metal needle handle 2 within a resin insertion tube 3 is described, in contrast to this, even when a needle handle 2 of an acupuncture needle 1 is made of a resin and an insertion tube 3 is made of a metal, by a high-frequency-induction-hearing method, the two can be secured and fixed to each other. In this case, arbitrary convex and concave portions or the like should be formed on an inner surface of the insertion tube 3 made of a metal.

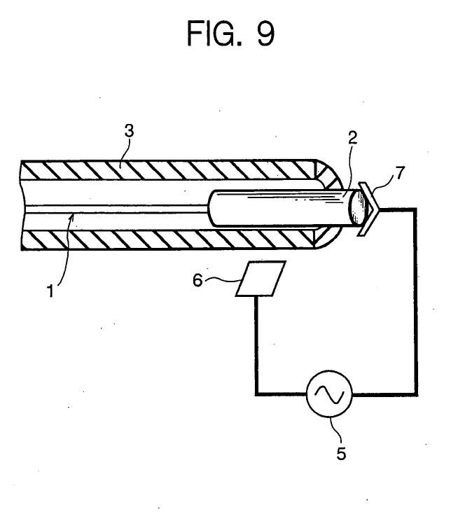

FIG. 9 is a schematic view showing a second method of securing an acupuncture needle to an insertion tube, according to the present invention. In the case of the second securing method, first, an acupuncture needle 1 having a metal needle handle 2 is inserted into a resin insertion tube 3 and the insertion tube 3 is arranged near an electrode 6 connected to an alternating-current power supply 5. Then, another electrode 7 connected to the alternating-current power supply 5 is connected to the metal needle handle 2 protruding from an opening end of the insertion tube 3. The electrode 7 has a clip configuration, for example, and is held by pinching an end portion of the needle handle 2 and thus the entire needle handle 2 made of a metal acts as an electrode.

In this state, when an alternating voltage is applied to these electrodes 6 and 7, a microwave is generated between the needle handle 2 connected to the electrode 7 and the electrode 6, and the microwave is irradiated to part of the insertion tube 3. Therefore, molecules of a resin in an area to which the microwave is irradiated vibrate, whereby friction heat is generated between the molecules and the insertion tube 3 itself can be momentarily heated. Then, by the heating of the insertion tube 3 itself, an area where an inner surface of the insertion tube 3 and a surface of the needle handle 2 are in contact with each other is melted and the needle handle 2 can be secured at the melted area 3a (refer to FIG. 1).

By such microwave heating, the insertion tube 3 itself which is an object to be heated can be momentarily heated, the heating temperature of the insertion tube 3 can be easily set by controlling the frequency of the alternating-current power supply 5 by an electric control circuit.

Even in a securing method by microwave heating such as the one described above, the acupuncture needle 1 never be detached from the insertion tube 3 by vibration or the like during testing, packaging, transporting, or the like, before being used for acupuncture treatment. Upon acupuncture treatment, by holding the insertion tube 3 and tapping an end portion of the needle handle 2, the acupuncture needle 1 can be released and detached from the insertion tube 3 with one hand.

Note that, in the case of the securing method by microwave heating, the shape can be any shape as long as the shape allows the surface of the needle handle 2 and the inner surface of the insertion tube 3 to come into contact with each other.

According to the present invention, an acupuncture needle having a metal needle handle can be secured within a resin insertion tube. In addition, by appropriately setting the shape, arrangement, or the like, of convex and concave portions formed on a surface of the needle handle of the acupuncture needle, the securing strength between the acupuncture needle and the insertion tube can be changed or an acupuncture needle with an insertion tube intended for single-use only can be manufactured. Consequently, industrial applicability is high.

I/(we) claims:

1. A method of securing an acupuncture needle to an insertion tube, comprising the steps of:
inserting the acupuncture needle having a needle handle made of any one of materials including a metal and a resin, into the insertion tube made of the other of the materials different from that of the needle handle;
arranging the insertion tube in a heating coil;
feeding a high-frequency alternating current to the heating coil to induction-heat one of the needle handle and the insertion tube, either of which is made of a metal; and
melting, by the heat, a resin in an area where a surface of the needle handle and an inner surface of the insertion tube are in contact with each other, to thereby secure the two to each other.

2. A method of securing an acupuncture needle to an insertion tube, comprising the steps of:
inserting the acupuncture needle having a metal needle handle into the insertion tube made of a resin;
arranging an electrode at a position outside the insertion tube while connecting another electrode to the needle handle;
applying irradiation of a microwave onto the insertion tube which is disposed between the electrodes to thereby allow an irradiated area to be heated; and
melting, by heat, an inner surface of the insertion tube to thereby effect fixing of the needle handle.

3. An acupuncture needle with an insertion tube in which the acupuncture needle having a needle handle made of any one of materials including a metal and a resin, is inserted into the insertion tube made of the other of the materials different from that of the needle handle, to thereby secure the two to each other, wherein
one of the needle handle and the insertion tube, either of which is made of a metal, is induction-heated by high-frequency-induction heating to heat and melt a resin in an area where a surface of the needle handle and an inner surface of the insertion tube are in contact with each other, thereby effecting fixing of the two to each other.

4. The acupuncture needle with the insertion tube according to claim 3, wherein the one of the needle handle and the insertion tube, either of which is made of a metal, has, formed thereon, concave portions or convex portions that annularly-arranged in a circumferential direction of a surface thereof.

5. The acupuncture needle with the insertion tube according to claim 3, wherein the one of the needle handle and the insertion tube, either of which is made of a metal, has, formed on a surface thereof, concave portions or convex portions that extend in an axial direction thereof.

6. The acupuncture needle with the insertion tube according to claim 3, wherein the one of the needle handle and the insertion tube, either of which is made of a metal, has, irregularly formed on a surface thereof, concave portions or convex portions in a dotted fashion.

7. An acupuncture needle with an insertion tube in which the acupuncture needle having a metal needle handle is inserted into the insertion tube made of a resin, to thereby secure the two to each other, wherein
the insertion tube is heated by microwave heating and an inner surface of the insertion tube is melted by heat, to thereby effect securing of the needle handle.
